(19) United States
(12) Patent Application Publication
(10) Pub. No.: US 2008/0045738 A1
(43) Pub. Date: Feb. 21, 2008

(54) Method for the Production of Amines from Compounds Comprising Carbodiimide Groups, by Hydrolysis With Water

(75) Inventors: Matthias Orschel, Munster (DE); Rainer Lomolder, Munster (DE); Stephan Kohlstruk, Dulmen (DE)

Correspondence Address: OBLON, SPIVAK, MCCLELLAND MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/591,975

(22) PCT Filed: Jan. 27, 2005
(86) PCT No.: PCT/EP05/50344

§ 371 Date: Jul. 20, 2007

(30) Foreign Application Priority Data
Mar. 9, 2004 (DE) 10 2004 011 320.3

(51) Int. Cl. C07C 271/24; C07C 211/35

(52) U.S. Cl. 560/25; 564/462

(57) ABSTRACT

The invention relates to a one-stage or multistage process for preparing mono-, di- and/or polyamines from compounds having carbodiimide groups and optionally also other groups of isocyanate chemistry by hydrolysis with water.

The invention relates to a one- or multi-stage process for preparing mono-, di- and/or polyamines from compounds having carbodiimide groups and optionally also other groups of isocyanate chemistry by hydrolysis with water.

Mono-, di- and/or polyamines are suitable, for example, as starting materials for preparing polyisocyanate polyaddition compounds, as starting materials in polycondensation processes or for preparing di- or polyisocyanate compounds. Aliphatic amines can be obtained by reacting alkyl halides or alcohols with NH₃ (ammonolysis), by reductive amination of ketones or aldehydes, by aminoalkylation (especially Mannich reaction), reduction of amides with lithium aluminum hydride, catalytic hydrogenation of nitrites, reduction of oximes with diborane or of azides with LiAlH4, and also by Hofmann degradation, Curtius rearrangement, Ritter reaction, Schmidt reaction or Gabriel synthesis. The aromatic amines are readily obtainable by reduction of the easily preparable nitro compounds (Ullmann's Encyclopedia of Industrial Chemistry, Wiley-VCH Verlag, 7^{th} Edition Release 2003). In addition, mono-, di- and/or polyamines can also be synthesized by acidic or alkaline, hydrolytic cleavage of urethanes, isocyanates and ureas [Houben-Weyl: Methoden der org. Chemie, 4. Auflage, Georg Thieme Verlag Stuttgart/New York (1957), 11/I, 948 ff.].

(Poly)carbodiimides are known and may be prepared, for example, selectively from substituted ureas, thioureas, carbamic esters, cyanamides, isocyanates, isothiocyanates or other carbodiimides [Houben-Weyl: Methoden der org. Chemie, 4. Auflage, Georg Thieme Verlag Stuttgart/New York (1987), E20/II, 1752; Houben-Weyl: Methoden der org. Chemie, 4. Auflage, Georg Thieme Verlag Stuttgart/New York (1983), E4, 888]. Owing to their reactivity, (poly)carbodiimides are used, for example, as stabilizers and promoters in polymer chemistry and to activate carboxylic acids in peptide synthesis. The reactions of (poly)carbodiimides with nucleophiles, for example water, alcohols and carboxylic acids, are known from the literature and afford the corresponding (poly)ureas, (poly)isoureas and (poly)acylureas [Wagner et al., Angew. Chem. (1981), 93, 855-866; Houben-Weyl: Methoden der org. Chemie, 4. Auflage, Georg Thieme Verlag Stuttgart/New York (1987), E20/II, 1756]. Specifically the addition of water to (poly)carbodiimides has been investigated in detail and in each case affords the corresponding urea [U.S. Pat. No. 2,938,892; DE 29 41 253; Lewis et al., Chem. Eur. (2002), 8, 1934; Tordini et al., J. Phys. Chem. A (2003), 107, 1188; Kurzer et al., Chem. Rev. (1967), 67, 107].

Up to the present time, there has been no known process for directly converting compounds having carbodiimide groups to the corresponding amines. It is therefore an object of the present invention to find such a process.

It has now been found that, surprisingly, amines can be prepared directly from the corresponding compounds having (poly)carbodiimide without isolating the ureas occurring as an intermediate.

The present invention thus provides a one- or multi-stage, continuous or batchwise process for preparing mono-, di- and/or polyamines from compounds having carbodiimide groups by hydrolysis with water.

The process for preparing mono-, di- and/or polyamines from compounds having carbodiimide groups and optionally also other groups of isocyanate chemistry by hydrolysis is effected by reacting (poly)carbodiimides with water, optionally also using an acidic or basic catalyst and/or optionally a solvent.

Compounds having carbodiimide groups which are used with preference are (poly)carbodiimides which have been modified with groups of isocyanate chemistry, for example aromatic, cycloaliphatic, (cyclo)aliphatic or aliphatic (poly)carbodiimides modified with urethane, isocyanate, amine, amide, (iso)urea, biuret, isocyanurate, uretdione, guanidine, formamidine, oxamidine, imidazoline, uretonimine and/or allophanate groups.

Preference is given to using the (poly)carbodiimides which are prepared from (poly)isocyanates, (poly)isocyanate derivatives or (poly)isocyanate homologues having aliphatic or aromatic isocyanate groups. Particular preference is given to using the (poly)carbodiimides which are prepared from the polyisocyanates, selected from 1,4-diisocyanatobutane, 1,6-diisocyanatohexane (HDI), 1,12-diisocyanatododecane, 1,4-diisocyaatocyclohexane, 1-isocyanato-5-isocyanatomethyl-3,3,5-trimethylcyclohexane (IPDI), bis(4-isocyanatocyclohexyl)methane (H12MDI), 1,3-bis(1-isocyanato-1-methyl)benzene (XDI), 1,3-bis(1-isocyanato-1-methylethyl)benzene (m-TMXDI), 2,4-diisocyanatotoluene (TDI), bis(4-isocyanatophenyl)methane (MDI), 1,6-diisocyanato-2,2,4(2,4,4)-trimethylhexane (TMDI), and where appropriate isomers higher homologues and technical-grade mixtures of the individual polyisocyanates.

Preference is given to using the compounds having the abovementioned carbodiimide groups to prepare polyamines selected from 1,4-diaminobutane, 1,6-diaminohexane, 1,12-diaminododecane, 1,4-diamionocyclohexane, 1-amino-5-aminomethyl-3,3,5-trimethylcyclohexane (IPDA), bis(4-aminocyclohexyl)methane (H12MDA), 1,3-bis(1-amino-1-methyl)benzene (XDA), 1,3-bis(1-amino-1-methylethyl)benzene (m-TMXDA), 2,4-diaminotoluene (TDA), bis(4-aminophenyl)methane (MDA), 1,6-diamino-2,2,4(2,4,4)trimethylhexane (TMDA) and where appropriate isomers, higher homologues and technical-grade mixtures of the individual polyamines.

The process is preferably carried out in such a way that the compounds having carbodiimide groups are reacted with an amount of water which is sufficient at least for the hydrolysis of the carbodiimide bonds and any groups of isocyanate chemistry which are also to be converted, at a temperature of from 0 to 400° C. and a pressure of from 0 to 500 bar. The mono-, di- and/or polyamines formed are isolated by suitable separation processes such as distillation, crystallization, extraction, sorption, permeation, phase separation or combinations thereof. The reaction may be effected using an acidic or basic, heterogeneous or homogeneous catalyst, and also optionally with a solvent or solvent mixture or both.

The amount of water required for the stoichiometric reaction is at least 2 mol of water per mole of carbodiimide group and a corresponding amount for the conversion of any additionally present groups of isocyanate chemistry. In principle, the amount of water used is not limited. However, preference is given to using from 2 to 100 times, more preferably from 5 to 80 times, most preferably 10 times the stoichiometric amount of water.

The process according to the invention may be carried out without or with solvent or solvent mixtures. The solvents used may be any common solvents; preference is given to using alcohols, particular preference to those alcohols which are formed in the hydrolysis of any urethane groups also present. The solvent may be used in any ratio, but preferably in a sufficient amount that the reaction mixture is present in monophasic form under the given reaction conditions. However, it is also possible to carry out the reaction in a biphasic or multiphasic mixture and thus to simplify the subsequent purification.

The process according to the invention may be carried out at temperatures of from 0 to 400° C., preferably from 150 to 300° C.

The process according to the invention may be carried out at a pressure of from 0 to 500 bar, preferably from 20 to 150 bar. Particular preference is given to working at the vapor pressure of the reaction mixture which is established at reaction temperature, which depends strongly on the composition.

The onset of the reaction can be recognized by the elimination of carbon dioxide. It is favorable to remove the carbon dioxide formed in the reaction from the reaction mixture, so that it is not available for secondary reactions (for example carbamic acid or salt formation). This discharge of carbon dioxide from the reaction mixture may be effected continuously or discontinuously and optionally with the use of a stripping gas, for example nitrogen.

The process according to the invention may be carried out continuously or batchwise in all common reactor systems, for example in stirred tank reactors, flow tube reactors, fluidized bed reactors, fixed bed reactors, bubble columns, reactive distillation reactors, microreactors or combinations or batteries of the reactors mentioned. The reaction may be carried out in one or more stages. In a multistage process, pressure and temperature and the amount of water and/or catalyst in the individual process steps are selected in such a way that the process is carried out in the first stage from the carbodiimide up to the urea und in the second stage up to the amine. There is no need to isolate and/or purify the intermediates.

The examples which follow serve to illustrate the process according to the invention, without it being restrictive thereto.

### EXAMPLE 1

### Conversion of dicyclohexylcarbodiimide to cyclohexylamine

500 g of dicyclohexylcarbodiimide are heated to 190° C. in an autoclave. Subsequently, 900 g of water are added with stirring from a reservoir heated to 190° C. The vapor pressure of the reaction mixture is established in the autoclave. During the reaction, the pressure rises further owing to the evolution of carbon dioxide. After a reaction time of 4 hours, the experiment is ended and the reaction mixture investigated by gas chromatography. A total of 178 g of cyclohexylamine are found, which corresponds to a theoretical yield of 37%, based on the dicyclohexylcarbodiimide used.

### EXAMPLE 2

### Conversion of dicyclohexylcarbodiimide to cyclohexylamine

500 g of dicyclohexylcarbodiimide are heated to 190° C. in an autoclave. Subsequently, 900 g of an aqueous, 0.25 molar sodium hydroxide solution are added with stirring from a reservoir heated to 190° C. The vapor pressure of the reaction mixture is established in the autoclave. During the reaction, the pressure rises further owing to the evolution of carbon dioxide. After a reaction time of 4 hours, the experiment is ended and the reaction mixture investigated by gas chromatography. A total of 298 g of cyclohexylamine are found, which corresponds to a theoretical yield of 62%, based on the dicyclohexylcarbodiimide used.

### EXAMPLE 3

### Conversion of dicyclohexylcarbodiimide to cyclohexylamine

The experiment is carried out in a similar manner to Example 2, except that the pressure in the autoclave is adjusted to 55 bar using nitrogen and a nitrogen stream of 50 g/h is passed through the reaction mixture over the entire reaction in order to continuously remove the carbon dioxide formed.

Overall, 399 g of cyclohexylamine are found, which corresponds to a theoretic yield of 83%, based on the dicyclohexylcarbodiimide used.

### EXAMPLE 4

### Conversion of

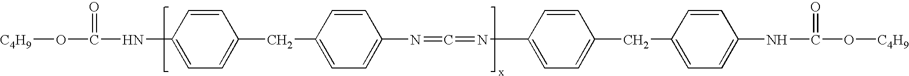

where x=4.7
to diaminodiphenylmethane

The reactant having the composition

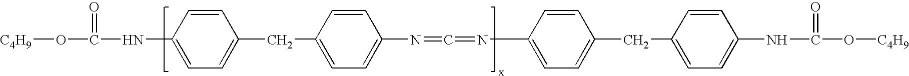

where x=4.7
is prepared according to U.S. Pat. No. 2,941,983 from diisocyanatodiphenylmethane and n-butanol (x=4.7; calculated from carbodiimide content and average molar mass). 50 g of this polycarbodiimide are heated to 230° C. in an autoclave with 400 g of n-butanol. Subsequently, 100 g of an aqueous, 0.25 molar sodium hydroxide solution are added with stirring from a reservoir heated to 230° C., and the pressure is adjusted to 55 bar using nitrogen. During the reaction, a nitrogen stream of 30 g/h is passed through the reaction mixture in order to continuously remove the carbon dioxide formed. After a reaction time of 4 hours, the experiment is ended and the reaction mixture investigated by gas chromatography. Overall, 33 g of diaminodiphenylmethane are found, which corresponds to a theoretic yield of 81%, based on the polycarbodiimide used.

### EXAMPLE 5

### Conversion of

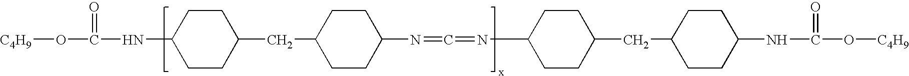

where x=1.1
to diaminodicyclohexylmethane

The reactant having the composition

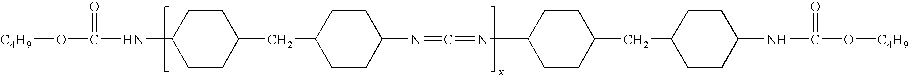

where x=1.1
is prepared in a similar manner to Example 4 from diisocyanatodicyclohexylmethane and n-butanol (x=1.1; calculated from carbodiimide content and average molar mass). 50 g of this polycarbodiimide are heated to 230° C. in an autoclave with 400 g of n-butanol. Subsequently, 100 g of an aqueous, 0.25 molar sodium hydroxide solution are added with stirring from a reservoir heated to 230° C., and the pressure is adjusted to 55 bar using nitrogen. During the reaction, a nitrogen stream of 30 g/h is passed through the reaction mixture in order to continuously remove the carbon dioxide formed. After a reaction time of 4 hours, the experiment is ended and the reaction mixture investigated by gas chromatography. Overall, 28 g of diaminodicyclohexylmethane are found, which corresponds to a theoretic yield of 87%, based on the polycarbodiimide used.

1. A one-stage, continuous or batchwise process for preparing di- and/or polyamines from compounds having carbodiimide groups by hydrolysis with water, the carbon dioxide formed being removed from the reaction mixture continuously or discontinuously, using a stripping gas.

2. The process of claim 1,
wherein
the compounds having carbodiimide groups which are used are (poly)carbodiimides which are prepared from (poly)isocyanates, (poly)isocyanate derivatives or (poly)isocyanate homologues having aliphatic or aromatic isocyanate groups.

3. The process of claim 1,
wherein
the compounds having carbodiimide groups which are used are (poly)carbodiimides which are prepared from the polyisocyanates selected from 1,4-diisocyanatobutane, 1,6-diisocyanatohexane (HDI), 1,12-diisocyanatododecane, 1,4-diisocyanatocyclohexane, 1-isocyanato-5-isocyanatomethyl-3,3,5-trimethylcyclohexane (IPDI), bis(4-isocyanatocyclohexyl)methane (H12MDI), 1,3-bis(1-isocyanato-1-methyl)benzene (XDI), 1,3-bis(1-isocyanato-1-methylethyl)benzene (m-TMXDI), 2,4-diisocyanatotoluene (TDI), bis(4-isocyanatophenyl)methane (MDI), 1,6-diisocyanato-2,2,4(2,4,4)-trimethylhexane (TMDI), or isomers thereof, higher homologues thereof and/or technical-grade mixtures of the individual polyisocyanates.

4. The process of claim 1,
wherein
(poly)carbodiimides are used which have been modified with groups of isocyanate chemistry.

5. The process of claim 4,
wherein
(poly)carbodiimides are used which have been modified with groups of isocyanate chemistry, selected from aromatic, cycloaliphatic, (cyclo)aliphatic or aliphatic (poly)carbodiimides which have been modified with urethane, isocyanate, amine, amide, (iso)urea, biuret, isocyanurate, uretdione, guanidine, formamidine, oxamidine, imidazoline, uretonimine and/or allophanate groups.

6. The process of claim 1,
wherein
the reaction is effected with an amount of water which is sufficient at least for the hydrolysis of the carbodiimide bonds and any groups of isocyanate chemistry which are also to be converted.

7. The process of claim 1,
wherein
the amount of water used is at least 2 mol of water per mole of carbodiimide group and a corresponding amount for the conversion of any additionally present groups of isocyanate chemistry.

8. The process of claim 7,
wherein
the amount of water used is from 5 to 100 times the stoichiometric amount, preferably from 5 to 50 times, more preferably 10 times the stoichiometric amount, based on the stoichiometric amount of water required to convert the carbodiimide groups and any additionally present groups of isocyanate chemistry.

9. The process of claim 1,
wherein
reaction is effected with an acidic or basic, heterogeneous or homogeneous catalyst or mixtures of acidic or basic heterogeneous homogeneous catalysts.

10. The process of claim 1,
wherein
the reaction is effected at a temperature of from 0 to 400° C.

11. The process of claim 10,
which
is carried out at temperatures of from 150 to 300° C.

12. The process of claim 1,
wherein
the reaction is effected at a pressure of from 0 to 500 bar.

13. The process of claim 12,
which
is carried out at a pressure of from 20 to 150 bar.

14. The process of claim 1,
wherein
the mono-, di- and/or polyamines formed are worked up by separation processes selected from distillation, crystallization, extraction, sorption, permeation, phase separation or combinations thereof.

15. The process of claim 1,
wherein
the reaction is effected with or without solvent.

16. The process of claim 15,
wherein
the solvent or solvent mixture used comprises alcohols, preferably those alcohols which are formed in the hydrolysis of any urethane groups also present.

17. The process of claim 1,
wherein
the working pressure is the vapor pressure of the reaction mixture which is established at reaction temperature.

18. The process of claim 1,
wherein
carbon dioxide formed in the process is removed from the reaction mixture continuously or discontinuously, using nitrogen.

19. The process of claim 1,
which
is carried out continuously or batchwise in reactor systems selected from stirred tank reactors, flow tube reactors, fluidized bed reactors, fixed bed reactors, bubble columns, reactive distillation reactors, microreactors or combinations or batteries of the reactors mentioned.

20. The process of claim 1,
wherein
polyamines selected from 1,4-diaminobutane, 1,6-diaminohexane, 1,12-diaminododecane, 1,4-diamionocyclohexane, 1-amino-5-aminomethyl-3,3,5-trimethylcyclohexane (IPDA), bis(4-aminocyclohexyl)methane (H12MDA), 1,3-bis(1-amino-1-methyl benzene (XDA), 1,3-bis(1-amino-1-methylethyl)benzene (m-TMXDA), 2,4-diaminotoluene (TDA), bis(4-aminophenyl)methane (MDA), 1,6-diamino-2,2,4(2,4,4)trimethylhexane (TMDA) and where appropriate isomers, higher homologues and technical-grade mixtures of the individual polyamines are prepared.
